(19) United States
(12) Patent Application Publication
(10) Pub. No.: US 2006/0148781 A1
(43) Pub. Date: Jul. 6, 2006

(54) Method and medicine for treating gastrointestinal disorder in a non-human mammal

(76) Inventor: James E. Lundeen, Cleveland Heights, OH (US)

Correspondence Address: RENNER OTTO BOISSELLE & SKLAR, LLP, 1621 EUCLID AVENUE, NINETEENTH FLOOR, CLEVELAND, OH, 44115, US

(21) Appl. No.: 10/970,149

(22) Filed: Oct. 21, 2004

Related U.S. Application Data
(60) Provisional application No. 60/518,715, filed on Nov. 10, 2003.
(60) Provisional application No. 60/518,718, filed on Nov. 10, 2003.
(60) Provisional application No. 60/518,719, filed on Nov. 10, 2003.

(51) Int. Cl. A61K 31/551; A61K 31/5415

(52) U.S. Cl. 514/220; 514/225.2

(57) ABSTRACT

A method and a medicine for treating a non-human mammal having a gastrointestinal disorder that includes fecal incontinence and/or irritable bowel syndrome are provided. The method includes administering a dose of the medicine to the mammal. The medicine includes a tricyclic antidepressant and a stool softener.

### CROSS-REFERENCE TO RELATED APPLICATIONS

This application claims priority to U.S. Provisional Patent Applications Ser. Nos. 60/518,715 filed on Nov. 10, 2003; 60/518,718 filed on Nov. 10, 2003; and 60/518,719 filed on Nov. 10, 2003, the disclosures of which are hereby incorporated by reference in their entirety.

### BACKGROUND

1. Field of Invention

The present invention generally relates to a method and a medicine for reducing or eliminating the undesirable affects of a gastrointestinal disorder. More particularly, the present invention relates to a method for reducing or eliminating symptoms of irritable bowel syndrome and/or fecal incontinence in a mammal. The present invention relates to a medicine for reducing or eliminating symptoms of irritable bowel syndrome and/or fecal incontinence in a mammal.

2. Discussion of Related Art

Mammals, such as cats and dogs, may be domesticated pets that live indoors. Domestication involves adapting the mammal to live in a human environment and to be of use to humans. But, sometimes the mammals may suffer from fecal incontinence. This is separate from training issues and the like associated with a pet with a healthy, functioning gastrointestinal system voiding its bowels indoors. Current treatment options for fecal incontinence range from exercise and dietary modification to drug therapy. But, currently there is no drug, medicine or pharmacologic treatment appropriate to all, or even most, suffering mammals.

With respect to diet, the mammal is not fed foods to which they possess a known sensitivity with respect to exacerbating the problem. With reference to drugs or medicines for the treatment of fecal incontinence, little research has gone into non-human drug studies. None of the medicinal candidates developed from the non-human drug studies have demonstrated sufficient efficacy to be of practical benefit to a majority of mammals. Drugs for the treatment of fecal incontinence include treatments directed to the gastrointestinal tract, and treatments directed to affective disorders mediated by the central nervous system (CNS), which are associated with fecal incontinence.

Drug treatment directed to the gastrointestinal tract includes antacids, anti-spasmodic agents, anti-diarrheal drugs, anti-inflammatory drugs such as glucocorticosteroids and NSAIDS, histamine-R2-blocking agents, antibiotics, and surgery.

Drugs having spasmolytic activity may be used to decrease intestinal motility. Amidinoureas, which reduce intestinal motility, may be useful for treating fecal incontinence. In addition to antispasmodic agents, compounds with other activities have been disclosed which may relieve the symptoms of fecal incontinence. Anti-diarrheal agents, such as loperamide, diphenoxylate, and codeine phosphate, have been used. They are unfortunately of little practical long-term benefit. Other anti-diarrheals include anti-cholinergics and smooth muscle relaxants, such as cimetropium bromide, pinaverium bromide, octilium bromide, trimebutine, and mebeverine.

Drugs designed to treat affective disorders mediated by the CNS include psychoactive drugs, such as anxiolytics and antidepressants. But, even if effective for a given mammal, psychoactive drugs are considered to have very limited and short-term utility. Non-selective excitatory opioid receptor antagonists have been identified as central nervous system treatments that affect the symptoms associated with fecal incontinence as well as irritable bowel syndrome. Non-selective excitatory opioid receptor antagonists include the tricyclic antidepressants, such as amitriptyline, imipramine, and doxepin have been used to treat irritable bowel syndrome, and may be effective due to the neuromodulatory and analgesic properties of these compounds. Because of the psychotropic properties, non-selective excitatory opioid receptor antagonist administration may be precluded for long-term care for chronic conditions, especially when prescribed for people rather than animals. In addition, the non-selective nature of the tricyclic antidepressants results in affectation of all five of the recognized muscarinic receptors and can cause undesirable side effects, such as dry eyes, dry mouth, etc.

In spite of the current treatments, compositions and methods used to reduce or eliminate symptoms associated with fecal incontinence no suitable long term, efficacious treatment or preventative has been identified. It would be desirable to have a medicinal composition or medicine having improved properties for the treatment of fecal incontinence and irritable bowel syndrome. It also may be desirable to have methods for the treatment of fecal incontinence and irritable bowel syndrome.

### SUMMARY

In one aspect, the present invention relates to a method for treating a mammal having a gastrointestinal disorder. The method includes administering a dose of the medicine to the mammal. The medicine includes a tricyclic antidepressant and a stool softener. The mammal may be, for example, a domesticated pet. In another aspect, the invention relates to the medicine for treating a mammal having a gastrointestinal disorder. The gastrointestinal disorder may include fecal incontinence, irritable bowel syndrome, or both.

Another aspect according to the invention relates to a process that includes interacting with muscarinic receptors in the mammal to reduce or eliminate fecal incontinence, and emulsifying oil and water into fecal matter using the surfactant to soften the stool, lubricating the fecal matter to facilitate passage of the stool, or both emulsifying and lubricating the fecal matter to both soften the stool and facilitate passage of the stool.

### BRIEF DESCRIPTION OF THE DRAWINGS

FIG. 1 is a schematic diagram showing a packaging configuration of a medicine comprising an embodiment in accordance with the invention; and

FIG. 2 is a schematic block diagram showing a method in accordance with the invention.

### DESCRIPTION OF PREFERRED EMBODIMENTS

The present invention generally relates to a method of treating a disorder of the gastrointestinal (GI) tract with a medicinal composition or medicine. The invention also relates to the medicine. The gastrointestinal disorder may be a chronic condition.

As used herein, a chronic condition refers to a condition that lasts for a substantial period or long time, and in some instances a chronic condition may not have an endpoint. Furthermore, chronic conditions may be continuous or recurring, and may reoccur regularly or irregularly. Gastrointestinal tract disorder includes fecal incontinence and irritable bowel syndrome. Unless specified or the context dictates otherwise, irritable bowel syndrome includes constipation-type irritable bowel syndrome (C-IBS), diarrhea-type irritable bowel syndrome (D-IBS), and alternating C-IBS and D-IBS. A medicinal composition (“medicine”) is a substance administered in the treatment of disorder; a remedial agent; and/or a remedy. An efficacious amount is an amount greater than zero that has a desired or desirable effect.

A method according to an embodiment of the invention includes administering a dose or a series of doses of the medicine to a mammal suffering from or presenting symptoms associated with a gastrointestinal (GI) disorder, such as fecal incontinence and/or irritable bowel syndrome. The medicine is described below, as is dosage information of the medicine. The fecal incontinence may be a result of, for example, one or more of nerve injury, a course of radiation treatments, a hemorrhoid surgery, a chemotherapy treatment, a compromised vascular supply to the bowel, malnutrition, diabetes, cancer, trauma, disease, or other, possibly unknown, sources. The nerve injury may be, for example, a spinal nerve injury or pelvic nerve injury. The compromised vascular supply to the bowel may be a result of, for example, a high cholesterol condition, collagen vascular disease, or a stroke of the bowel mesenteric artery.

In one embodiment, the medicine includes a tricyclic antidepressant and one or both of a stool softener and a fecal lubricant. Tricyclic antidepressants may be used alone or in combination and may include amitriptyline, clomipramine, desipramine, imipramine, doxepin, and nortriptyline, and derivatives and pharmaceutically acceptable salts thereof. Unless otherwise specified or indicated by context, “stool softener” will herein collectively include both stool softener and fecal lubricant for ease of referral.

In one embodiment, the tricyclic antidepressant includes imipramine (5-[3-(dimethylamino) propyl]-10,11-dihydro-5H-dibenzazepine), which is shown structurally below, or an active metabolite thereof—such as desmethylimipramine.

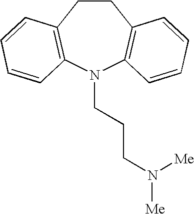

In another embodiment, the tricyclic antidepressant includes imipramine HCl. Imipramine hydrochloride is available for commercial sale as TOFRANIL from Mallinckrodt Inc. (St. Louis, Mo.). As used throughout, reference to dosage of imipramine generally will be to an equivalent amount of imipramine HCl. Also, unless specified, dosage values are in units of milligrams.

In yet another embodiment, the tricyclic antidepressant includes imipramine Pamoate (5-[3-(dimethylamino) propyl]-10,11-dihydro-5H-dibenzazepine 4,4-methlyenebis-(3-hydroxy-2-napthoate) (2:1 ratio of pamoate to imipramine). imipramine pamoate is commercially available as TOFRANIL-PM from Mallinckrodt Inc. (St. Louis, Mo.).

For an adult animal, a total daily dosage of imipramine in a medicine according to an embodiment of the present invention may be in a range of from about 5 mg/day to about 100 mg/day, about 10 mg/day to about 25 mg/day, about 25 mg/day to about 50 mg/day, or about 50 mg/day to about 75 mg/day. Here and elsewhere, range limitations may be combined, for example, a range may be from about 10 mg/day to about 50 mg/day.

Alternatively, in one embodiment the total daily dosage may be based on weight. According to an embodiment of the present invention a total daily dosage of imipramine in a medicine may be in a range of from about 0.1 milligram/kilogram body weight/day (mg/kg/day) to about 2.5 mg/kg/day, about 0.2 mg/kg/day to about 1.2 mg/kg/day, about 0.5 mg/kg/day to about 2.0 mg/kg/day, about 0.5 mg/kg/day to about 0.75 mg/kg/day, about 0.75 mg/kg/day to about 1.25 mg/kg/day, or about 1.25 mg/kg/day to about 2.0 mg/kg/day.

Because younger animals may have a relatively higher glomerular filtration rate (GFR) the dosage may need to be adjusted upward to accommodate such, rather than downward as seen in anti-depression treatment. In one embodiment, a dosage for a younger animal may be up four times greater than an adult dosage, or up to about 400 mg/day. For elderly, infirm, or smaller than average-sized mammals a total daily dosage amount may be adjusted downward, for example, in a range of from about 5 mg to about 50 mg.

In one embodiment, the tricyclic antidepressant may be taken concomitant or concurrent with the stool softener. In one embodiment, the tricyclic antidepressant may be taken at a time different than the stool softener. The regimen for taking the medicine, or components or portions thereof, is discussed further below.

The stool softener used herein is distinguished from laxatives. Laxatives include bulk, osmotic and stimulant-type. Bulk laxatives include soluble and insoluble fiber. Soluble fiber can include psyllium husks and is commercially available as METAMUCIL from Procter & Gamble Inc. (Cincinnati, Ohio). Insoluble fiber can include wheat bran. Osmotic laxatives are not absorbed and function by pulling water into the colon via osmotic action (e.g., magnesium hydroxide, such as PHILLIP'S MILK OF MAGNESIA, which is commercially available from Bayer Corporation (Pittsburgh, Pa.)). Stimulant laxatives interfere with absorption of water from the colon lumen and motility of fecal material therethrough.

By way of contrast, a stool softener may act to emulsify water and/or oil into fecal matter and thus soften the consistency. A fecal lubricant may act by lubricating the fecal matter and allowing it to pass though the colon with a reduced amount of friction. Suitable surfactants include anionic surfactants. Other suitable surfactants may include nonionic surfactants, cationic surfactants, and amphoteric surfactants. In one embodiment, the stool softener includes bis(2-ethylhexyl) sulfosuccinate sodium salt (“docusate sodium”), which is commercially available from Purdue Phama L.P. (Stamford, Conn.) as COLACE. Other suitable metal salts of sulfosuccinate also are useful, and the metal may be potassium, calcium and the like. PERICOLACE (which is a tradename for docusate plus casanthrol), sodium dodecylsulfate (SDS), sodium cholate, sodium deoxycholate (DOC), N-lauroylsarcosine sodium salt, lauryldimethylamine-oxide (LDAO), and cetyltrimethyl ammoniumbromide (CTAB) may be used in embodiments according to the invention.

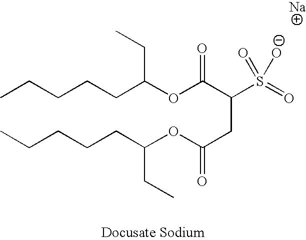

The fecal lubricant may include, for example, commercially available mineral oil or liquid paraffin. The stool softener and fecal lubricant may be used alone and in combination with each other. In combination, the stool softener can emulsify the fecal lubricant into the stool.

In one embodiment according to the invention, the stool softener may be used in an efficacious amount at dosage levels of less than 200 mg/day. In one embodiment, the dosage of stool softener may be greater than 200 mg/day, and may be used in an amount of up to about 300 mg/day, or up to about 400 mg/day.

Alternatively, the amount of the stool softener may be determined with reference to body weight. In one embodiment, the total daily dosage may be in a range of from about 1 mg/kg/day to about 4 mg/kg/day. In one embodiment, the total daily dosage may be in a range of from about 1.0 mg/kg/day to about 2.0 mg/kg/day, from about 2.0 mg/kg/day to about 3.0 mg/kg/day, or from about 3.0 mg/kg/day to about 4.0 mg/kg/day.

The amount taken or total daily dosage may be selected with reference to predetermined factors. Such factors may include, for example, seasonal changes (e.g., dehydration being more prevalent in summer months may result in a higher incidence of constipation-type symptoms, et cetera), aging, the natural course of the gastrointestinal disorder, stress inducing situations, and others that may affect the occurrence or severity of symptoms of the gastrointestinal disorder.

The dosage amount of tricyclic antidepressant to stool softener may be expressed as a ratio or a proportion. In one embodiment, the ratio of tricyclic antidepressant to stool softener is in a range of from about 1:80 to about 3:1, from about 1:12 to about 1:6, from about 1:4 to about 1:3, from about 1:2 to about 1:1, or from about 2:1 to about 3:1. In one embodiment, the ratio may be preselected based on weight, symptom severity, symptom type, symptom frequency, dietary considerations, type of tricyclic antidepressant and stool softener, dose regimen, administration method, environmental considerations, other or additional medications, and the like. In one embodiment, the ratio may be selected based on individual responsiveness, dietary considerations, environmental considerations, side effects, aggravating conditions such as stress level, other or additional medications, and the like.

With reference to form of the medicine, at least a portion of the medicine may be in the form of a pill, capsule, gelcap, a coated or chewable tablet, an ingestible liquid admixture, an enema or suppository, an intravenous solution or an intramuscular injectable liquid.

For pills, capsules, gelcaps, tablets, and the like, suitable packaging includes multi-dose packages, such as blister packs. The blister packs may contain dosages of the medicine according to the present invention.

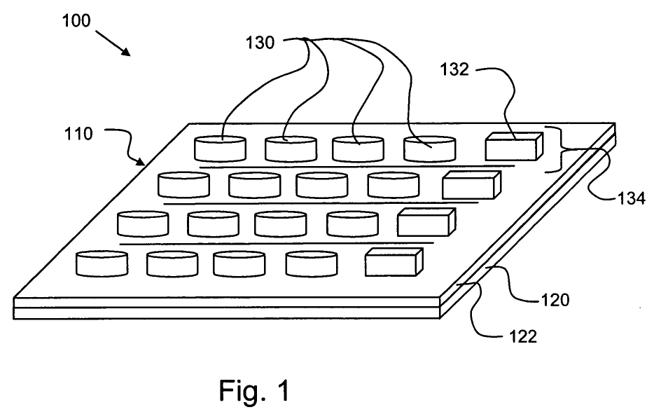

With reference to FIG. 1, a packaged treatment regimen 100 showing an embodiment according to the invention includes a blister pack 110. The blister pack 110 has a base layer 120 secured to a bottom surface of a top layer 122. The top layer 122 defines storage blisters, and the base layer 120 can operate to seal the blisters to releasably contain doses of the medicine, or portions of the medicine. The blisters in the illustrated embodiment define differing shapes merely for the purpose of ease of differentiation. In the embodiment shown, stool softener may be housed in the blisters labeled 130, and the tricyclic antidepressant is housed in the blister labeled 132. A row or strip 134 may equal a total daily dose of the medicine. Because in the illustrated embodiment, the total daily dose includes four portions of stool softener (at, for example, 75 mg each) and one portion of tricyclic antidepressant (at, for example, 25 mg), there are correspondingly four blisters 130 for housing the stool softener and one blister 132 for housing the tricyclic antidepressant. Thus, the stool softener may be taken four times a day for 300 mg/day total daily dose, and the tricyclic antidepressant may be taken once a day for 25 mg/day total daily dose. Furthermore, the tricyclic antidepressant may be taken with one of the stool softener doses or at another time, as desired. The strip 134 is one of four shown on the blister package 110, which is a four day supply of medicine. The blister package 110 may have instructions printed thereon that indicate what the dosage regimen may be, and, optionally and/or additionally, directions for varying portion dosage with reference to symptomology or exacerbating conditions.

In other embodiments (not shown) the tricyclic antidepressant and stool softener portions may include dosages having differing amounts for different total daily dosages, may have differing numbers of doses for the same or different total daily dosages, and may have doses that include both the tricyclic antidepressant and the stool softener in a single form (such as a pill containing both the tricyclic antidepressant and the stool softener). Further, other embodiments according to the invention may have the tricyclic antidepressant and/or the stool softener in a form other than pill, gel cap, and the like, and may not be amenable to blister packaging. Suitable packaging may be selected based on the form of the tricyclic antidepressant and the stool softener, and whether the tricyclic antidepressant and the stool softener are admixed or physically separate.

Administering the dose may include selecting an entry method into the animal based on the form of the medicine. For example, if the imipramine is formed as gelcap, and the sodium docusate is an ingestible liquid, the imipramine may be swallowed while the sodium docusate may be imbibed or drank. And the sodium docusate may be taken either at about the same time as the imipramine or at a different time during a day. Imipramine and sodium docusate may be combined in a single capsule, in which case the capsule may be taken once a day or as part of a series of capsules taken throughout a day depending on the dosage amount in each capsule.

The tricyclic antidepressant may be delivered by a first method (such as oral delivery), while the stool softener may be administered via a different method. The enema or suppository may contain the stool softener and may be administered in a conventional manner.

For orally administrable embodiments in which at least one component or portion of the medicine is taken orally, masking agents may be used. For example, edible carriers, such as food, may be used to enhance palatability of the medicine or medicine component. Dosages of the medicine may be hidden within food to facilitate administration. In one embodiment, the food is selected to have a pharmacological effect.

In one embodiment, the medicine may contain additional material either admixed or separate from the tricyclic antidepressant, the stool softener, or both. For example, the medicine may contain a skeletal muscle relaxant, a narcotic, or a proton pump inhibitor, and may further include a suitable pharmaceutical excipient, diluent, or carrier selected with regard to the intended route of administration and standard pharmaceutical practice. Suitable skeletal muscle relaxants include cyclobenzaprine hydrochloride, which is also classified as a tricyclic antidepressant and is commercially available from McNeil Corporation (Fort Washington, Pa.) as FLEXERIL. Cyclobenzaprine hydrochloride may be combined in the medicine according to the invention. A useful dose of cyclobenzaprine hydrochloride may be about 10 milligrams a day.

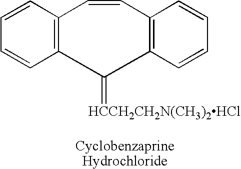

Suitable narcotics include opioid agonists include PERCOCET (oxycondone plus acetaminophen), which is commercially available from Endo Laboratories, Inc. (Chadds Ford, Pa.). Suitable proton pump inhibitors include omeprazole or 5-methoxy-2-[[(4-methoxy-3,5-dimethyl-2-pyridinyl) methyl]sulfinyl]-1H-benzimidazole, which is commercially available from AstraZeneca LP (Wilmington, Del.) as PRILOSEC, and lansoprazole, which is commercially available from TAP Pharmaceutical Products Inc. (Lake Forrest, Ill.) as PREVACID.

In one embodiment, the medicine further includes a beta-blocker, such as atenolol, which is commercially available from Medley Pharmaceuticals, Ltd (Maharashtra, India) as TENORMIN. Atenolol is a synthetic, betal-selective (cardioselective) adrenoreceptor blocking agent or “beta-blocker”, that may be chemically described as benzeneacetamide, 4-[2′-hydroxy-3′-[(1-methylethyl) amino]propoxy]benzeneacetamide. Atenolol may block the action of the sympathetic nervous system. Because the sympathetic nervous system controls or influences the pace of the heart beat, blocking the action of these nerves can reduce the heart rate. Atenolol may reduce the force of heart muscle contraction, lower blood pressure, and may affect fecal incontinence and symptoms associated with irritable bowel syndrome, such as bowel frequency. Where tachycardia may be caused, for example, as a result of the action of the tricyclic antidepressant, a beta-blocker such as atenolol may be used to maintain the heart rate in a desired range.

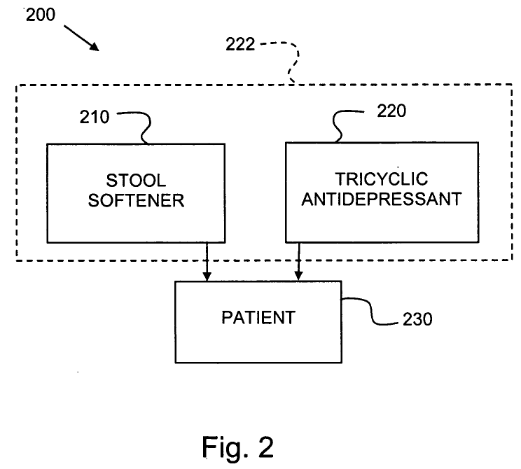

With reference to FIG. 2, a method according to the present invention is shown as a block diagram 200. A stool softener 210 and a tricyclic antidepressant 220 comprise a medicine 222. The stool softener 210 and the tricyclic antidepressant 220 are administered to a mammal 230 suffering from a fecal incontinence.

### EXAMPLES

Embodiments according to the invention are illustrated in the following examples. In particular, the treatment of fecal incontinence by methods and with medicines according to the present invention is shown.

### Example 1

A female cat presents with fecal incontinence. The mammal weighs 1 kilogram and is treated with a daily dose of medicine, which includes 0.1 mg/kg of imipramine pamoate and 0.5 mg/kg stool softener. After several days of daily treatment via oral administration, the mammal has control of fecal incontinence and does not appear to show distress relating to irritable bowel syndrome.

### Example 2

A male dog presents with fecal incontinence and irritable bowel syndrome. The mammal weighs 5 kg and is treated with a daily dose of medicine, which includes 1.6 mg/kg of imipramine pamoate and 2 mg/kg stool softener. After several days of daily treatment via oral administration, the mammal has control of fecal incontinence and does not appear to show distress relating to irritable bowel syndrome.

### Example 3

A female dog presents with fecal incontinence. The mammal weighs 10 kg and is treated with a daily dose of medicine, which includes 1.6 mg/kg of imipramine pamoate and 2 mg/kg of stool softener. After several days of daily treatment via oral administration, the mammal has control of fecal incontinence.

### Example 4

A female dog presents with irritable bowel syndrome related constipation and gastric distress. The mammal weighs 10 kg and is treated with a daily dose of medicine, which includes 0.2 mg/kg of imipramine pamoate and 1 mg/kg of stool softener. After several days of daily treatment via oral administration, the mammal has normal bowel function and does not appear to have gastric distress.

The processes and embodiments described herein are examples of compositions, systems and methods having elements corresponding to the elements of the invention recited in the claims. This written description may enable those skilled in the art to make and use embodiments having alternative elements that likewise correspond to the elements of the invention recited in the claims. The intended scope of the invention thus includes other compositions, systems and methods that do not differ from the literal language of the claims, and further includes other compositions, systems and methods having equivalents of, or with insubstantial differences from, the literal language of the claims.

What is claimed is:

1. A method of treating a non-human mammal having a gastrointestinal disorder comprising fecal incontinence, the method comprising:
administering a dose of a medicine to the mammal having the gastrointestinal disorder, the medicine comprising
a tricyclic antidepressant, and
a stool softener.

2. The method as defined in claim 1, wherein the gastrointestinal disorder is fecal incontinence.

3. The method as defined in claim 1, wherein the gastrointestinal disorder further comprises irritable bowel syndrome, and the gastrointestinal disorder is a result of one or more of a nerve injury, a disease, cancer, or a compromised vascular supply to the bowel.

4. The method as defined in claim 3, wherein the nerve injury is a spinal nerve injury or pelvic nerve injury.

5. The method as defined in claim 1, wherein the tricyclic antidepressant is present in an efficacious amount in a range of less than about 200 milligrams per day.

6. The method as defined in claim 5, wherein the tricyclic antidepressant is present in an efficacious amount in a range of less than about 75 milligrams per day.

7. The method as defined in claim 1, wherein the stool softener is present in an amount in a range of greater than about 200 milligrams per day.

8. The method as defined in claim 7, wherein the stool softener is present in an amount in a range of greater than about 300 milligrams per day.

9. The method as defined in claim 1, wherein the tricyclic antidepressant comprises imipramine hydrochloride, imipramine pamoate, a pharmacologically acceptable salt of imipramine, or combinations of two or more thereof.

10. The method as defined in claim 1, wherein the mammal is a non-elderly adult mammal.

11. The method as defined in claim 1, wherein the mammal is a cat or a dog.

12. The method as defined in claim 1, wherein the stool softener comprises a surfactant, a fecal lubricant, or a combination of surfactant and fecal lubricant.

13. The method as defined in claim 12, wherein the surfactant comprises an anionic surfactant.

14. The method as defined in claim 13, wherein the anionic surfactant comprises docusate sodium.

15. The method as defined in claim 1, wherein the gastrointestinal disorder is a chronic condition, and the administering is performed over an extended period of time corresponding to a treatment of the chronic condition.

16. The method as defined in claim 1, further comprising disposing the tricyclic antidepressant and the stool softener adjacent to each other or spaced from each other, or admixing with each other, during packaging.

17. The method as defined in claim 1, further comprising forming at least a portion of the medicine as a pill, capsule, gelcap, an ingestible liquid admixture, transdermal patch, an inhalable powder or mist, an enema or suppository, a coated or chewable tablet, a chewable gum, an intravenous solution, or an intramuscular injectable liquid, and administering comprises selecting an entry method into the mammal based on the form of the medicine.

18. The method as defined in claim 1, further comprising administering the tricyclic antidepressant and the stool softener substantially simultaneously or sequentially relative to each other.

19. The method as defined in claim 1, further comprising varying amounts of the tricyclic antidepressant and the stool softener over a course of treatment in response to severity of symptoms of the gastrointestinal disorder.

20. The method as defined in claim 1, further comprising administering to the mammal a beta-blocker that is responsive to reduce tachycardia, a skeletal muscle relaxant, a narcotic, a proton pump inhibitor, or two or more thereof.

21. The method as defined in claim 1, wherein the tricyclic antidepressant is administered in an efficacious amount in a range of about 0.1 mg/kg/day to about 2.5 mg/kg/day.

22. The method as defined in claim 21, wherein the tricyclic antidepressant is administered in an efficacious amount in a range of about 0.5 mg/kg/day to about 2 mg/kg/day.

23. The method as defined in claim 1, wherein the tricyclic antidepressant to stool softener ratio is in a range of from about 1:80 to about 3:1.

24. The method as defined in claim 23, wherein the tricyclic antidepressant to stool softener ratio is in a range of from about 1:4 to about 1:3.

25. The method as defined in claim 1, wherein the stool softener is administered in an efficacious amount in a range of about 1 mg/kg/day to about 4 mg/kg/day.

26. The method as defined in claim 25, wherein the stool softener is administered in an efficacious amount in a range of about 2 mg/kg/day to about 3 mg/kg/day.

27. A medicinal composition for treating a mammal having a gastrointestinal disorder comprising fecal incontinence, the composition comprising:
a tricyclic antidepressant, and
a stool softener.

28. The composition as defined in claim 27, wherein the gastrointestinal disorder further comprises irritable bowel syndrome.

29. The composition as defined in claim 27, wherein the gastrointestinal disorder is a result of one or more of a nerve injury, a disease, cancer, or a compromised vascular supply to the bowel.

30. The composition as defined in claim 29, wherein the nerve injury is a spinal nerve injury or a pelvic nerve injury.

31. The composition as defined in claim 27, wherein the tricyclic antidepressant is present in an efficacious amount in a range of less than about 200 milligrams per total daily dose.

32. The composition as defined in claim 31, wherein the tricyclic antidepressant is present in an efficacious amount in a range of less than about 75 milligrams per total daily dose.

33. The composition as defined in claim 27, wherein the stool softener is present in an amount in a range of greater than about 200 milligrams per total daily dose.

34. The composition as defined in claim 27, wherein the stool softener is present in an amount in a range of greater than about 300 milligrams per total daily dose.

35. The composition as defined in claim 27, wherein the tricyclic antidepressant comprises imipramine hydrochloride, imipramine pamoate, a pharmacologically acceptable salt of imipramine, or combinations of two or more thereof.

36. The composition as defined in claim 27, wherein the mammal is a non-elderly adult.

37. The composition as defined in claim 27, wherein the mammal is a dog or a cat.

38. The composition as defined in claim 27, wherein the stool softener comprises a surfactant, a fecal lubricant, or a combination of surfactant and fecal lubricant.

39. The composition as defined in claim 38, wherein the surfactant comprises docusate sodium.

40. The composition as defined in claim 27, further comprising a beta-blocker that is responsive to reduce tachycardia, a skeletal muscle relaxant, a narcotic, a proton pump inhibitor, or two or more thereof.

41. The composition as defined in claim 27, wherein medicine is configured as fractional dosage amounts, the fractional dosage amounts being operable to effect variations in a total daily dosage amount of the tricyclic antidepressant and of the stool softener over a course of treatment.

42. The composition as defined in claim 27, wherein the tricyclic antidepressant and the stool softener in the medicine are configured for packaging to be adjacent to each other in each dose or admixed with each other in each dose for administration substantially simultaneously with each other; or
the tricyclic antidepressant and the stool softener in the medicine are packaged separate from each other for administration substantially simultaneously, sequentially, or alternating periodically with each other.

43. The composition as defined in claim 27, wherein at least a portion of the medicine is in the form of a pill, capsule, gelcap, an ingestible liquid admixture, transdermal patch, an oral or nasal inhalable powder or mist, an enema or suppository, a coated or chewable tablet, a chewable gum, an intravenous solution, or an intramuscular injectable liquid.

44. The composition as defined in claim 27, wherein the medicine is in the form of a plurality of co-packaged dosages of the medicine, and each dose comprises a portion of a daily dosage amount, wherein
each dose comprises the tricyclic antidepressant and the stool softener, or
a first portion of the plurality of dosages comprises the tricyclic antidepressant and not the stool softener, and a second portion of the plurality of dosages includes the stool softener and not the tricyclic antidepressant, and doses of the first portion and the second portion are administrable to form a total daily dose.

45. The composition as defined in claim 27, wherein the tricyclic antidepressant is administered in an efficacious amount in a range of about 0.1 mg/kg/day to about 2.5 mg/kg/day.

46. The composition as defined in claim 45, wherein the tricyclic antidepressant is administered in an efficacious amount in a range of about 0.5 mg/kg/day to about 2 mg/kg/day.

47. The composition as defined in claim 27, wherein the tricyclic antidepressant to stool softener ratio is in a range of from about 1:80 to about 3:1.

48. The composition as defined in claim 47, wherein the tricyclic antidepressant to stool softener ratio is in a range of from about 1:4 to about 1:3.

49. The composition as defined in claim 27, wherein the stool softener is administered in an efficacious amount in a range of about 1 mg/kg/day to about 4 mg/kg/day.

50. The composition as defined in claim 49, wherein the stool softener is administered in an efficacious amount in a range of about 2 mg/kg/day to about 3 mg/kg/day.

51. A treatment kit for a mammal having a gastrointestinal disorder comprising irritable bowel syndrome, fecal incontinence, or both, the kit comprising:
a plurality of doses of a medicine, the medicine comprising:
a tricyclic antidepressant, and
a stool softener comprising one or more of a surfactant and a fecal lubricant.

52. The kit as defined in claim 51, further comprising an instruction set comprising directions for administering the medicine, the instruction set comprising dosage amounts, dosing schedules, or a combination thereof.

53. A process for treating a gastrointestinal disorder comprising irritable bowel syndrome, fecal incontinence, or both in a mammal, the process comprising:
causing interaction with muscarinic receptors in the mammal to reduce or eliminate the gastrointestinal disorder by affecting a stool of the mammal, and
delivering into fecal matter an oil and water by emulsification using a surfactant to soften the stool of the mammal,
delivering into fecal matter a fecal lubricant to facilitate passage of the stool, or
emulsifying and lubricating the fecal matter to both soften the stool and facilitate passage of the stool,
wherein the emulsifying, lubricating, or emulsifying and lubricating occurs in the bowel of the mammal.
